(19) United States
(12) Patent Application Publication
(10) Pub. No.: US 2026/0054056 A1
(43) Pub. Date: Feb. 26, 2026

(54) INJECTRODE SYSTEM

(71) Applicant: NEURONOFF, INC., Cleveland, OH (US)

(72) Inventors: Manfred Franke, Cleveland, OH (US); Stephan Nieuwoudt, Cleveland, OH (US); Sachit Kshatriya, Cleveland, OH (US); Derrick Liu, Cleveland, OH (US); Amy Howe, Cleveland, OH (US)

(73) Assignee: NEURONOFF, INC., Cleveland, OH (US)

(21) Appl. No.: 19/104,394

(22) PCT Filed: Aug. 24, 2023
(86) PCT No.: PCT/US2023/031099

§ 371 Date: Feb. 18, 2025

Related U.S. Application Data
(60) Provisional application No. 63/400,709, filed on Aug. 24, 2022.
(60) Provisional application No. 63/400,725, filed on Aug. 24, 2022.

(51) Int. Cl. A61N 1/05; A61N 1/36

(52) U.S. Cl. CPC A61N 1/0504; A61N 1/36003

(57) ABSTRACT

A system for stimulating a tissue target in a body with an injectable electrode, either fully or partially implanted, may employ a protection device to prevent over-voltage or over-current from an external pulse generator (EPG) to the injectable electrode. Current is supplied from the EPG transcutaneously to the fully implanted injectable electrode and percutaneously to the partially implanted injectable electrode by various electrical connectors.

### PRIORITY STATEMENT

This application is a National Phase entry of International Application PCT/US2023/031099 and claims priority to, and the full benefit of, U.S. provisional patent application No. 63/400,709 filed Aug. 24, 2022, and U.S. provisional patent application No. 63/400,725 filed Aug. 24, 2022.

### FIELD OF THE INVENTION

The present invention relates to the use of injectable electrodes formed by helical wire rope structures suitable for injection.

### BACKGROUND OF THE INVENTION

This application incorporates PCT/US21/33007 (the '007 application) filed May 18, 2021 (US national application Ser. No. 18/278,160 entered on Aug. 21, 2023) as if set forth entirely herein. The present application uses reference numbers as used in the '007 application.

An injectable electrode is described in the '007 application.

As used herein, an “injectable electrode” is the same as, and interchangeable with, a helical wire rope structure.

### SUMMARY

The present disclosure is directed to a device and system incorporating a helical wire structure rope electrode as described herein, in the '007 application, and with additional aspects disclosed in the present application. In various embodiments in which the injectable electrode is fully implanted, a system for stimulating a tissue target in a body comprising a helical wire rope structure 1 electrode having a hollow core 5 extending along a length with first and second ends 3 configured for loading into a dispenser in a substantially linear form extending along the length for injection, and further configured for bending or bunching into a plurality of irregular shapes 8 in the body, a first of the irregular shapes at the first end near a tissue target and a second of the irregular shapes in a subcutaneous area and the helical wire rope structure electrode configured to transmit electricity passively along all the length, and an external pulse generator (EPG) 32A for generating an electrical waveform, and configured to transmit electricity through an electrical path across the skin to the second irregular shape at the first end in the subcutaneous area. In a related embodiment, the EPG provides the waveform transcutaneously through at least two interfacing points to at least one bunched anchor/irregular shape of the implanted helical wire rope structure, and optionally at least one additional helical wire rope structure, thereby steering current to minimize off-target stimulation. In another embodiment, the system further comprises a protective unit 91, 92 to prevent current or voltage above a safe level, with the protective unit located within the EPG or within the electrical path from the EPG to the helical wire rope structure electrode.

The embodiments of the injectable electrode disclosed in the '007 application are fully implanted. One of these embodiments is an injectable device for delivering electrical current to a tissue target in a body, the device configured for loading into and injection from a dispenser to be fully implanted in the body, the device comprising a helical wire rope structure having an initial length and first and second ends and comprising coils of at least one twisted wire rope, the twisted wire rope comprising strands of conductive wire configured so that some of the strands can loosen partially from and be spaced apart from the coils, the helical wire rope structure from the first end to the second end having a hollow core, being substantially linear along all of the initial length when loaded inside the dispenser, having capabilities of stretching and flexing, having capabilities of bunching and bending up to 180 degrees upon injection and being configured to allow formation near the tissue target of a first irregular shape near the first end to self-anchor the first end without tines, hooks or sutures and to provide an electrical charge interface to the tissue target, and being surrounded between the first and second ends by nonconductive coating comprising at least one gap or hole. As further described in the '007 application, in one embodiment the bunching and the bending of the injectable electrode allows formation in a subcutaneous region of a second irregular shape near the second end and the second end and/or a substantially linear portion of the helical wire rope structure are configured to collect electrical current by capacitive and resistive coupling transcutaneously with an external pulse generator (EPG) for generating an electrical waveform, the EPG 32A being outside the body and transmitting current which is transmitted passively along the helical wire rope structure to the tissue target 51, such as a nerve.

Disclosed in the present application are also usages and configurations of the injectable electrode which are not fully implanted (the “percutaneous injectable electrode”), and which are incorporated into a system. In one embodiment a system for stimulating a tissue target comprises a helical wire rope structure 1 having a hollow core 5 extending along a length with first and second ends 3 configured for loading into a dispenser in a substantially linear form extending along the length for injection of a portion of the length into a body, and further configured for bending or bunching into at least one irregular shape 8 at any location along the length inside the body for self-anchoring without tines, hooks or sutures, the second end configured to extend as an external tail 99 beyond the skin 37, and the helical wire rope structure electrode configured to transmit electricity passively along all the length, an external pulse generator (EPG) 32A for generating an electrical waveform, and a lead from the EPG and an electrical connection to the external tail. In a related embodiment the EPG provides the waveform through at least two interfacing points on the external tail on the helical wire rope structure, and optionally a second helical wire rope structure, transcutaneously, thereby steering current to minimize off-target stimulation. In another embodiment the system further comprises a protective unit to prevent current or voltage above a safe level wherein the protective unit is located within the EPG, at an interface between the EPG and the lead, within the lead, at a connection between the lead and the helical wire rope structure electrode, or within the helical wire rope structure electrode.

In various embodiments of a system with the fully implanted injectable electrode or the percutaneous injectable electrode, the system also comprises one or more over-voltage protector 91 or over-current protector 92 to prevent voltage or current above a safe level. These protective units are discussed further herein.

### BRIEF DESCRIPTION OF THE FIGURES

FIG. 1 is an image of an injectable electrode and, on the right end, the wire rope before it is coiled.

FIG. 2A is an image of the entire length of an injectable electrode after being taken off of the guidewire, also known as a mandrel, as shown in FIG. 2B.

FIG. 3A is a close up of the end of the injectable electrode as in FIGS. 2A and 2B. FIG. 3B labels the individual coils in the injectable electrode.

FIGS. 4A and 4B are additional images of the injectable electrode on a guidewire/mandrel.

FIGS. 5A and 5B are closeup images of a portion of injectable electrodes comprising different wire widths.

FIGS. 6A and 6B are closeup images of injectable electrodes on a mandrel.

FIGS. 7A, 7B, 8, 9A, 9B and 10 are images of injectable electrodes as cut to show the interior including the hollow core, except for 7B in which the core remains filled with the guidewire/mandrel before it is removed after manufacture.

FIGS. 11 and 11A are schematics of fully implanted injectable electrodes with and without a central anchor/irregular shape between the ends. The coils and texture of the injectable electrodes are not depicted.

FIG. 12 is a schematic of protection units of an embodiment of circuits for over-voltage and over-current.

FIGS. 13A, 13B, and 13C are schematics of EPGs with the over-voltage protection unit built within the EPG, and how they connect to patch electrodes.

FIGS. 14, 15 and 16 are schematics for other locations for over-voltage protection units.

FIG. 16A illustrates a prototype of an over-voltage protection unit.

FIG. 17A is a schematic of a combined over-voltage and over-current protection unit built within the EPG, and 17B is the same outside the EPG.

FIG. 18 is a section view of the system herein with a partially implanted percutaneous injectable electrode connected with a conductive metal band secured in an adhesive strip, and the EPG connected. FIG. 18A shows the top of the adhesive strip and conductive metal band, and 18B shows the underside facing the body and the percutaneous injectable electrode.

FIG. 19 is a section view similar to FIG. 18, except that the second end of the injectable electrode has been implanted to secure the external tail.

FIGS. 20A-D are images of injectable electrodes showing holes or gaps in the coating.

FIG. 21 shows multiple images of a time sequence of ingress of fluid into a coated injectable electrode through one or more holes or gaps.

FIG. 22 shows images showing MRI results of injectable electrodes implanted in agar samples.

FIG. 23 shows images showing twisting and coiling patterns for lang lay and regular lay.

FIGS. 24A-24C are images of various configurations of the twisting and coiling of the injectable electrode.

FIGS. 25A-25F are schematics showing the greater bending capability of a lang lay (FIGS. 25A-25C) injectable electrode compared to regular lay (FIGS. 25D-25F).

### DETAILED DESCRIPTION

As used in this application, a “strand” 4 can be lengths of a continuous wire skeined or separate and individual lengths of wire 22. For an injectable electrode comprising separate strands of wire twisted together into a rope, the wire ends may be crimped so that they do not irritate surrounding tissue, or they may be gathered and coated in a polymer on the circumference of the end but leaving the wire ends exposed to conduct energy. Individual wire ends may also be heated so they form a mass.

The injectable electrode possesses a general shape and pattern of a helix made of at least one wire rope which comprises a number of strands. Although the general shape and pattern of the helix is predictable among all embodiments given standardized manufacturing techniques, the placement of an individual strand and the actual shape and dimension of a single coil in the helix may be random or irregular, but only in this sense within a general shape or pattern. In the same way, a bunching anchor 8 from a helical structure with a given wire composition and standardized manufacturing may be random or irregular, but only in this sense within a general shape and pattern.

The injectable electrode, in various embodiments, comprises conductive wire selected from the group consisting of platinum-iridium, gold, silver, platinum, stainless steel, titanium, titanium-nickel, iridium, tungsten, platinum-tungsten and other metal alloys such as MP35N, a cobalt-nickel-chromium alloy with molybdenum added for corrosion resistance. Wires comprising the above metals are readily available commercially in the 2-300 micron diameter range, and wires of other diameters are also suitable for some embodiments. The final selection of material and diameter for a particular embodiment is dependent on patient biocompatibility and desired tensile (mechanical) and electrical properties for the particular application and embodiment. It is also dependent on optimum force supplied by the injectable electrode onto the tissues against which it is pressed, because mechanical forces due to differences in material compliance or flexibility from any implanted injectable electrode influence formation of encapsulation tissue. Wires of biocompatible metals have different mechanical properties, such as hardness, and electrical properties, such as conductivity, and the potential effects for heating of the wires during the conduction of electrical current. The metal composition of the wires can be varied to introduce desired physical properties. The deployment process partially unwinds some of the strands 4 from the main helical structure and pushes them between 1 to 200 microns away that is mechanically distant enough from the helical structure 1 to be mechanically free to move with the tissue and have less encapsulation between that strand and the native tissue. Also, the strands which partially unwind are very flexible (because they are so thin and can move even more easily than a coil of the helical structure) and can be even closer to the tissue target 51 and help with conduction of energy as the electrical field reduces over distance. In this way the deployment process partially unfolds the tightly wound wire rope 22 forming the helical structure and helps to create more flexibility and thus better mechanical matching between the helix and the surrounding tissue. This is a helical macrostructure with a multi-strand microstructure.

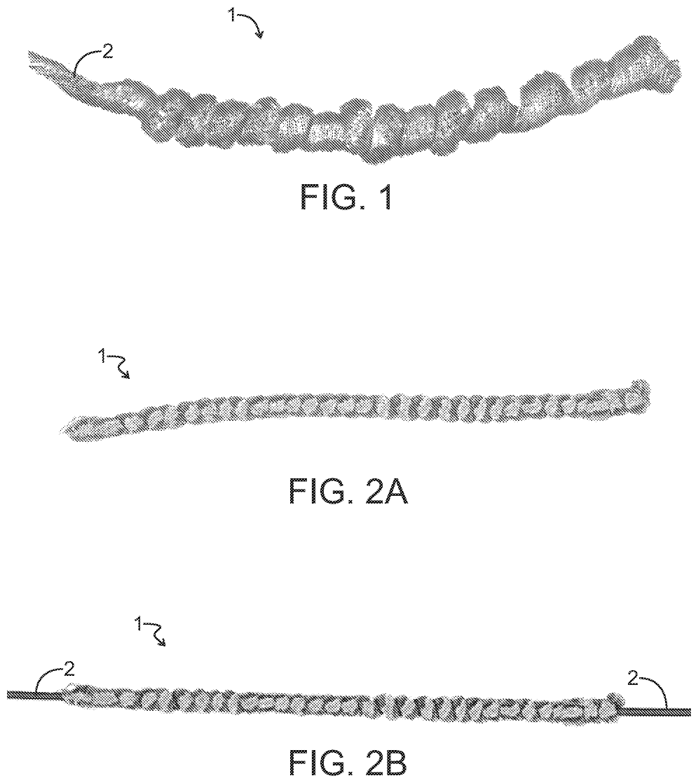

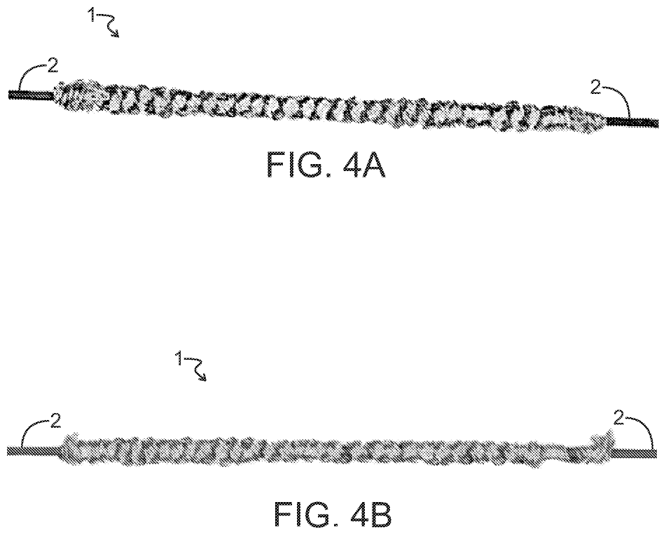

FIG. 1 shows a conductive wire rope 22 (pre-helical structure) at the left and the helical wire rope structure, or injectable electrode 1, at center and right, after removal of the guidewire/mandrel around which the wire rope was coiled. The end 3 of the helical wire structure (shown in FIG. 3A) is round as this embodiment is made of strands formed from one continuous length of wire. Multiple wire ropes may comprise the helical structure, depending on the diameter of wire used for the wire ropes, and the desired flexibility of the helical wire rope structure. These multiple wire ropes are either electrically connected or insulated from one another, for which the latter embodiment results in a multi-contact electrode.

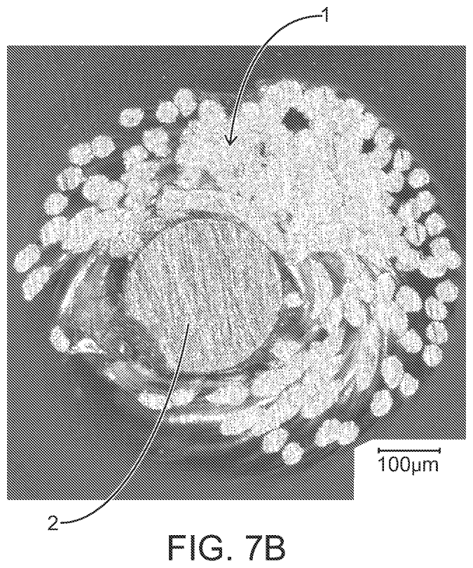

After the guidewire/mandrel 2 has been removed from the injectable electrode, there is a hollow core 5 (shown in FIG. 7A) surrounded by the inner diameter 17 of the helix 1 which allows for liquid, gel or gas delivery/transport through the helical structure while still inside the dispenser 10. This ability to transfer liquid, gel or gas into the body or out of the body (i.e. injection or suction) may be used during hydro dissection and formation or widening of the void 12 within or nearby the interfacing tissue target right before or during the deployment into the body of the helical wire structure.

Another aspect of the hollow core is that it can be filled with interstitial fluid in the body, as discussed herein regarding MRI compatibility.

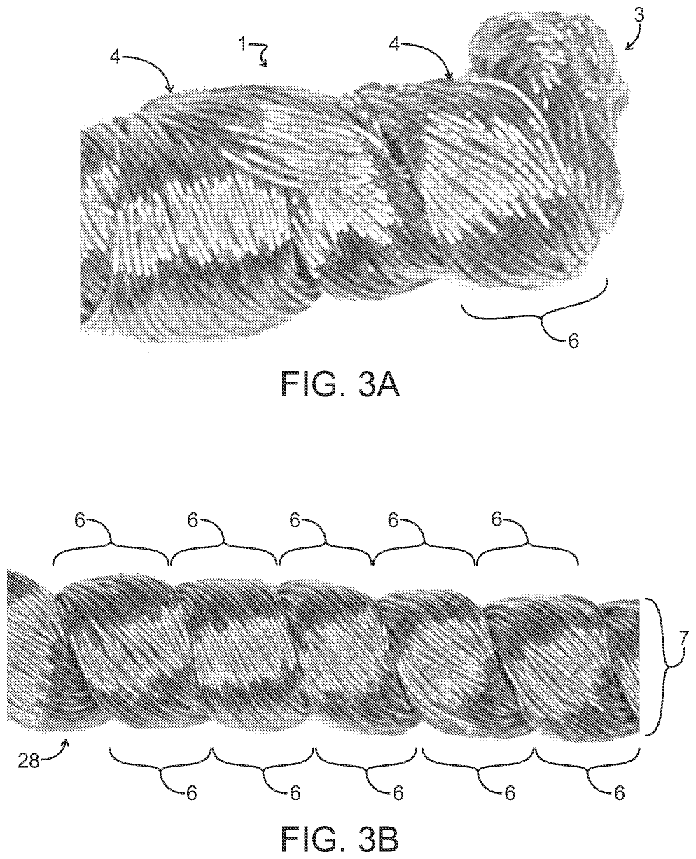

FIG. 2A is an image (15.6×) of one embodiment of the injectable electrode with the lang lay helical wire structure ex vivo after removal of a guidewire/mandrel, and FIG. 2B is the same structure (7.4×) with the guidewire/mandrel 2. In this embodiment the wire rope is 100 strands of 25 diameter micron wire and is wrapped around a 0.25 mm guidewire/mandrel with an approximate outer diameter of 0.75 mm. Overall length is approximately 2 cm and is made from 6 meters of continuous wire.

FIG. 3A is a close up of an end of the injectable electrode in FIGS. 2A & 2B showing an end 3, here rounded because the wire in the wire rope is continuous resulting in no sharp ends. FIG. 3B is a close up (100×) of a middle portion of the electrode in FIGS. 2A & 2B, and shows five full coils 6 (or turns) with two additional coils partially cropped out of the image. The outer diameter 7 of the injectable electrode's helical wire structure is also shown.

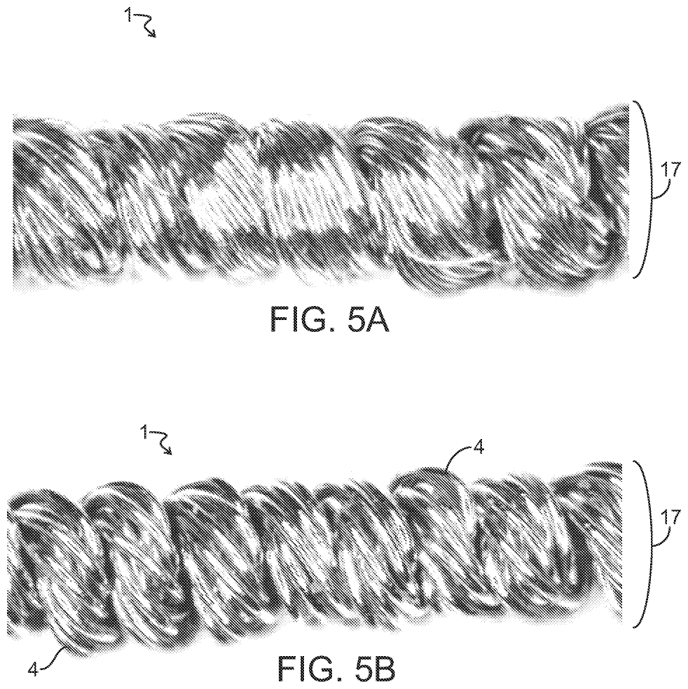

FIG. 4A is an image (14.3×) of one embodiment of the injectable electrode with the lang lay helical wire structure 1 comprising 50 strands of 25 micron diameter wire and 5 wires of 75 micron diameter wire. FIG. 4A shows an embodiment with the helical wire structure comprising 10 strands of 75 micron diameter wire.

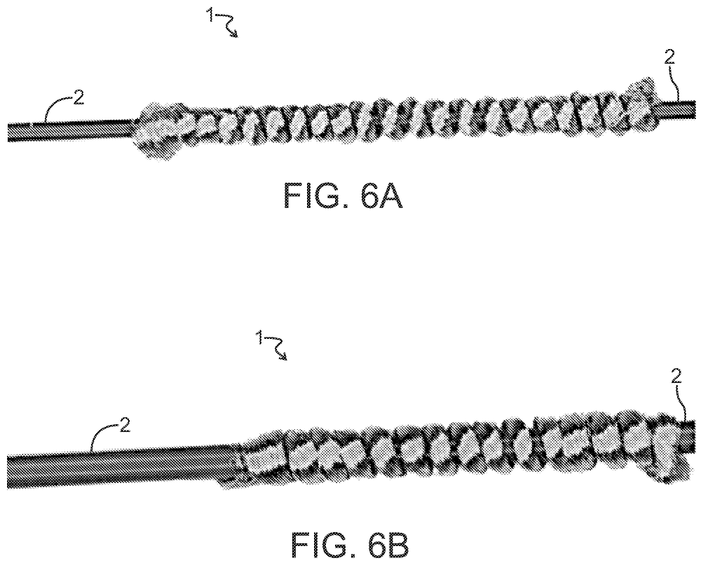

FIG. 5A is a closer view (100×) of the injectable electrode 1 with different wire diameters in FIG. 4A, and the coil where the arrow is pointing has a different mix of wire diameters than those to the left of it. FIG. 5B is a closer view (100×) of the electrode with wire strands of 75 micron diameter in FIG. 4B. The average outer diameter 7 for the electrodes in FIGS. 5A & 5B are the same.

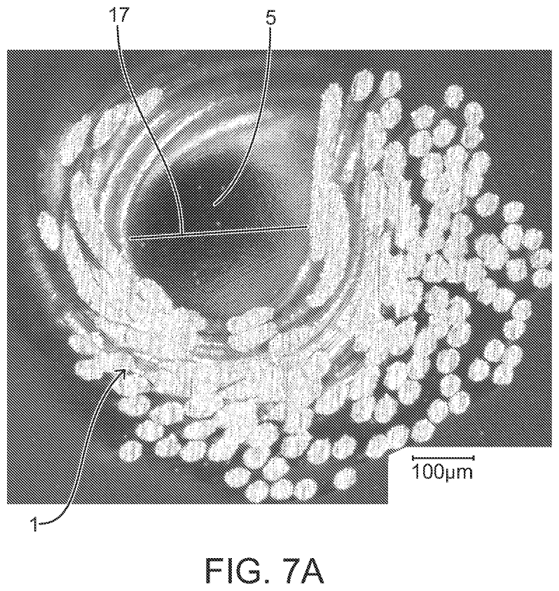

FIG. 6A is an image of an embodiment of the lang lay injectable electrode 1 comprising 100 strands of 25 micron diameter wire, each with a guidewire/mandrel 2 of 0.45 mm diameter, and FIG. 6B shows an embodiment on a guidewire/mandrel 2 of 0.8 mm.

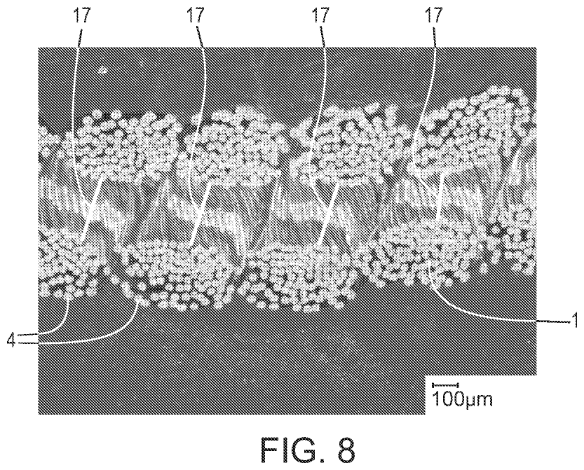

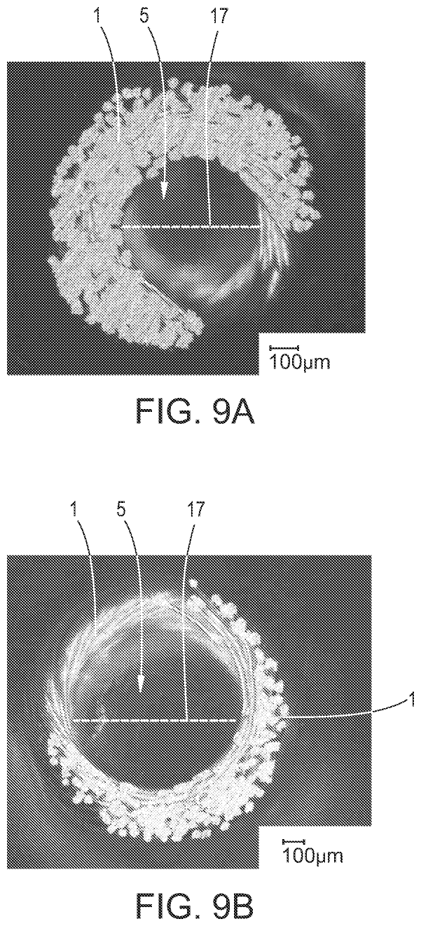

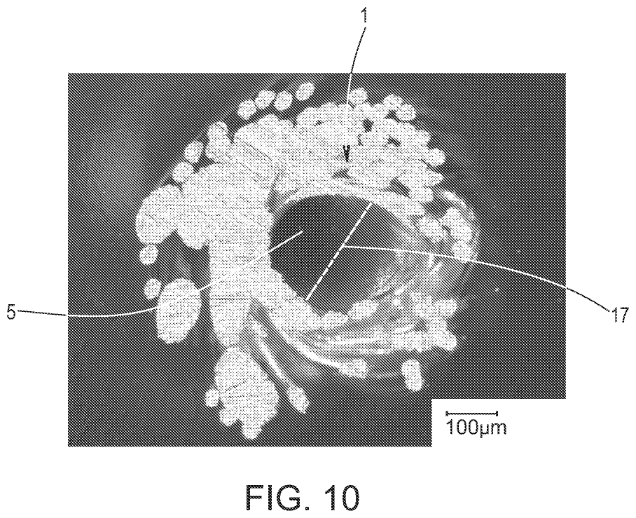

FIG. 7A is an image (300×) of a latitudinal cross-section of an electrode comprising a lang lay helical wire structure 1 comprising a wire rope comprising 100 strands of 25 micron diameter wire. Some distortion of the structure exists because of the cutting process. This figure shows the hollow core 5 defined by the inner diameter 17, here approximately 0.25 mm. FIG. 7B is an image of the same electrode before the 0.25 mm guidewire/mandrel 2 was removed. The guidewire/mandrel was chipped slightly on the lower left by the cutting process. FIG. 8 is an image (150×) of a longitudinal cross section of the same electrode as in FIG. 7A showing the inner diameter 17 defining the hollow core 5. Again, here, there is some distortion resulting from cutting, as in all of the cross-section figures herein. FIG. 9A is an image (200×) of an electrode similar to FIGS. 7A, 7B, and 8 but manufactured on a larger guidewire/mandrel, 0.45 mm, and FIG. 9B shows an electrode formed on a guidewire/mandrel of 0.8 mm. In FIGS. 7A, 8, 9A, & 9B the guidewire/mandrels have been removed. FIG. 10 is an image (300×) of a latitudinal cross-section of an electrode comprising a helical wire structure comprising a wire rope comprising 50 strands of 25 micron diameter wire and 5 strands of 75 micron diameter wire, with a hollow core 5.

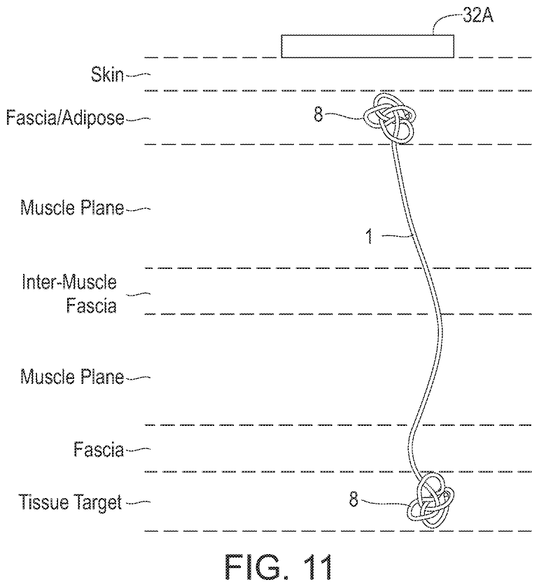

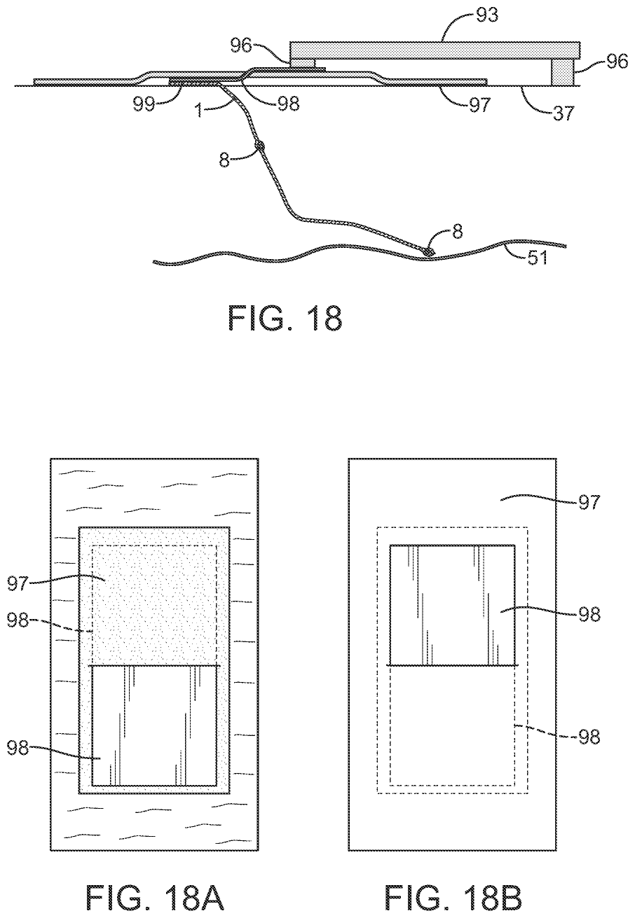

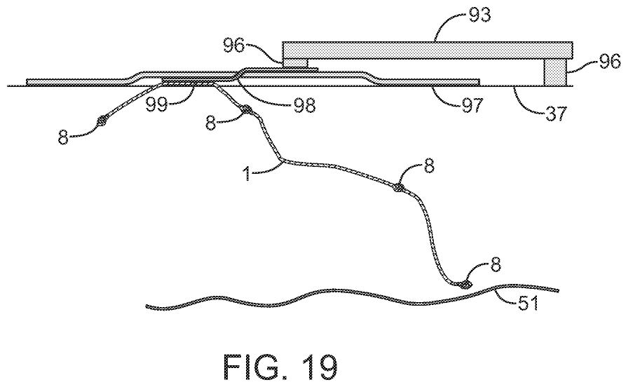

FIG. 11 is a conceptual diagram of the helical wire structure electrode 1 as implanted with a bunching anchor 8 at a tissue target 51 (as labeled in FIGS. 18 and 19) such as a nerve, with a second bunching anchor 8 in the subcutaneous fascia/adipose region, which is able to receive transcutaneous transmission of electrical current from a transcutaneous electrical nerve stimulation (TENS) unit 32 on the skin surface. The bunching anchor serves as a connector pad to enhance reception of energy either transcutaneously from a probe 29 (as shown in FIG. 11B of the '007 application) or from a TENS unit or similar. FIG. 11 does not attempt to show the helical structure or the exact nature of the bunching anchor 8, but just the general positioning of the helical wire structure within the tissue. The connector pad is the same as the bunching anchor 8 shown throughout this specification, the connector pad for a fully implanted injectable electrode being close enough to the surface of the body to enable collection of electric current transcutaneously from a TENS or percutaneously by a probe 29 transmitting energy (the latter as shown in FIG. 11B of the '007 application).

The injectable electrode properties allow it to (1) flex easily when it comes in contact with tissue during the delivery when being pushed out of the needle, (2) flex so that it does not injure the neural tissue when being pushed out of the needle, (3) flex so that it bends and bunches and forms to self-anchor the coated and uncoated regions, in many embodiments in three locations, one on each end and an uncoated portion or more between the ends and (4) unzip easily by being able to let go from the tissue orthogonally to the direction of the length of the injectable electrode. This is accomplished by having a (1) significantly large hollow core (created by the guidewire/mandrel earlier), and (2) a comparably small rope diameter (when compared to the dimensions of the hollow core). The ratio of the hollow core diameter to the rope diameter as well as the resulting helix to rope diameter matter.

Data for an injectable electrode from a low-force during unzipping of entire device is 250 um/910 um=0.27 and 250 um/950 um=0.26, and the hollow core is large enough to flex, unzip reliably at low forces. Unzipping and flexing even with a smaller mandrel (thus smaller hollow core) of about 150 um or 200 um. Given this, use of a 200 um mandrel to produce a 910-950 um outer diameter, then the ratio of hollow core to outer helix diameter would be at least ⅕ instead of ¼. The ratio can also be phrased as the inner diameter of the helix divided by the outer diameter of the helix as the inner diameter of the hollow core of the helix is defined by the guidewire/mandrel. That ratio of inner diameter to outer diameter of the helix throughout the entire length of the device end to end in one embodiment is >¼ or, in another embodiment, >⅕).

In another embodiment, an injectable electrode without a coating has an outer diameter of 280-300 um (at ends of injectable electrode and at gaps in coating), but with a coating an outer diameter of 330 to 350 um. The is 250 um with an outer helix diameter of 910 to 950 um. The ratio of rope to guidewire/mandrel anywhere along the length is from 280 um/250 um=1.12:1 to 1.4:1, or 350 um/250 um. A ratio of helix to rope anywhere along the length can be from (950 um/350 um=2.7 to 910 um/280 um=3.25. A ratio of helix outer diameter to guidewire/mandrel (hollow core) can range from (910 um/250 um=3.64 to 950 um/250 um=3.8)

Thus, in some embodiments, a ratio of helix outer diameter to hollow core (or guidewire/mandrel) is <4. (smaller than 4). This means that at least one quarter of outer diameter is hollow core along the entire length of the injectable electrode. Stated differently, at least one quarter of the outer diameter of the helix is the hollow core.

One aspect which has not been known in the art is protection against voltage or current outside a range for the patient which is safe and comfortable. Presented here is a voltage or current limiting fuse, meaning a hardware solution and not software alone, as part of a system with either an IPG or an EPG.

While it is common for TENS units to have an output stage that has a maximum voltage compliance, a maximum voltage or current regulator, these devices are designed to be able to interface with a broad spectrum of patients and there are no patient specific hardware based limitations in existence. An example are TENS units that supply electrical pulses to nerves close to the skin, generally utilizing hydrogel patch electrodes to apply the TENS unit currents and voltages to the skin of a patient where the TENS unit itself has a maximum voltage compliance of e.g. 50 V or 100 V. Patients are physically able to increase the output voltages and current on the TENS unit to levels that are able to cause pain by electrically activating small (e.g. nociceptive) pain fibers in the skin near the hydrogel patch electrodes. A goal of treatment is for the current to pass by these pain fibers in the skin and activate sensory B-fibers and A-motor fibers in the skin and the subcutaneous tissue. TENS unit manufacturers thus rely on the patient's own pain response to limit an over-voltage or over-current application to deep A-motor and sensory B-fibers. Similarly, there are devices that utilize a TENS to supply electrical energy to an implanted lead wire, the lead wire having the ability to conduct some of the subcutaneously received electrical energy to a nerve target deeper inside the body; these systems also lack a patient-specific over-voltage and over-current protection as the coupling between the TENS and the implanted lead seem to be insufficient to couple large enough currents to the deeper nerve structures within the body. Likewise, for IPG based systems, software is generally being used to limit the maximum voltage and/or current applied to a specific electrode implanted in a given patient. That is, an existing system includes a bare metal contact interfacing with a liquid electrolyte material with ionic conductors present, and they transmit whatever current is provided to them from the lead. For commonplace TENS units targeting tissue near the skin, current or voltage protection is not needed. To assist with an implanted pickup device under the skin, as a connector to tissues much deeper than the skin, a greater focus of energy crossing the skin is desirable, with protection from excess voltage and current.

For existing TENS units, then, there is no essential need to provide an overvoltage or over-current protection as the skin separates the TENS electrode, a large surface area interface, from a subcutaneous nerve, generally a small surface area tissue. Also, pain fibers in the skin provide a sensory input to the user to avoid applying too large of a voltage or current to the deeper nerves by transmitting a pain perception to the brain from the skin underneath the TENS patch electrode before pain fibers or other fibers are generally activated too much in a deep nerve.

For the fully implanted injectable electrode as presented in the '007 application, however, a subcutaneous region of the injectable electrode transfers current to a tissue target deeper within the body, and especially when the injectable electrode passes through or tightly around the tissue target, and so a focusing effect may occur. This effectively results in the tissue target, such as a nerve, to be exposed to a higher current and voltage than in the absence of an injectable electrode.

The degree and impact of the focusing effect depend on the following factors:
A. Currents and voltages applied as electrical waveforms over time on the outside of the body. A variety of elements may be used to describe the waveform, among them are amplitudes, frequencies, pulse widths, alternating component only or direct current components (or offsets), pause times, bursts of pulses, and the like.
B. Dimensions of the EPG and dimensions of the active surface area of the subcutaneous region of the injectable electrode.
C. Alignment in X/Y direction between the EPG and the active surface area of subcutaneous region of the injectable electrode, in essence, overlap or lack of overlap.
D. Distance in Z direction between the EPG and the active surface area of subcutaneous region of the injectable electrode, z.e., separation distance (as in between two capacitor plates).
E. Amount of active surface area on the distal end of the injectable electrode in contact with or close proximity to a target tissue.
F. Quality and preparation of the patient skin and its effect on surface contact impedance.
   The focusing effect may enable the activation of a nerve relatively far removed from the subcutaneous tissue while currents passing through the skin are barely noticeable.

In addition to the focusing effect, there is a need to prevent unintentional overvoltage or over-current resulting from a device failure or the influence of an externally applied electrical field while at least the electrodes of the system are in contact with the body and in close vicinity to the implanted injectable electrode.

Generally, any pulse generator utilizing software may be treated by medical device regulators as having the risk of a software failure that, if not mitigated through hardware means, may pose higher risk to the patient than a system with an additional hardware mitigation in place. A typical hardware mitigation can be an over-voltage and/or over-current protection on the outside of the EPG 32A or between the EPG and the patient.

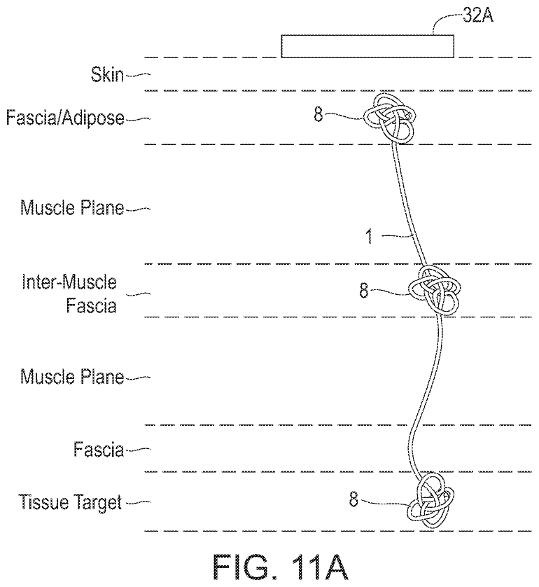

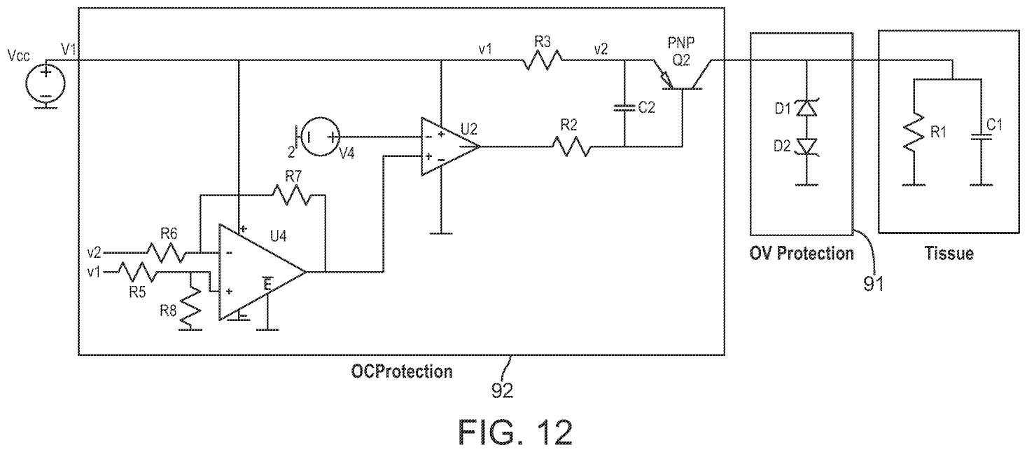

FIG. 12 is a schematic showing a basic approach to over-voltage and overcurrent protection for the injectable electrode. The box labeled 91 in FIG. 12 is one type of an over-voltage protector comprising two diodes 91A placed in an anti-parallel configuration such that the voltage applied to the patient is applied in parallel to the two anti-parallel diodes. The term “anti-parallel” describes the two diodes as facing each other (FIG. 13A) or away from each other (FIG. 13C) while being electrically in series with each other, meaning the forward opening and the backward breakdown voltages for the two diodes add together to form a joint maximum breakdown voltage at which the majority of the current will start passing through the diodes instead of passing through the patient. In various embodiments, the two diodes will be of the same type and thus the joint maximum breakdown voltage will be the same in both directions, so that a charge balanced symmetrical waveform applied voltage will shunt equally through the diodes instead of reaching the patient. In certain cases and in alignment with the waveform generator, two different diodes are combined to form different sum breakthrough voltages in cathodic versus anodic stimulation directions. Additionally, other embodiments comprise more than two diodes, and transistors and resistors are added to allow for more sophisticated over-voltage protection circuitry. In one example (FIG. 13B), a capacitor C is added to capture only a certain portion of an over-voltage, just as a voltage spike at the beginning of an otherwise rectangular voltage signal and return it back to the circuit during the charge balancing phase of the waveform. The box labeled 92 in FIG. 12 is one type of an over-current protector comprising a variety of circuit elements such as resistors R and capacitors C that allow for currents beyond a reasonably assessed level to pass through this path in parallel to the patient instead of passing through the patient themselves. All of these devices can be placed at different locations.

The EPG 32A shown in FIGS. 13A-13C comprises a signal generation unit 93 which generates an initial unmodified output such as a stimulating waveform to a signal output unit 94, or junctions, buttons or connectors on the outside of the signal generation unit. The signal then passes through leads 95 which connect to patch electrodes 96, one of which connecting to the external tail 99 and one to the skin as a return electrode (as shown in FIGS. 18 and 19).

There are several locations along the signal generation-delivery pathway where protective measures can be implemented. The five main locations are (a) at the stimulator level (inside the signal generator), (b) at the interface between stimulator and leads (outside the stimulator), (c) at the lead level, (d) at the interface between the lead and the electrode, and (e) the electrode level.

There are several reasons for this protection. For example, if software malfunctions and an output on the signal generator is disrupted or remains on when not intended, the patient could be given a stimulating charge continuously or at unintended levels. The hardware solutions 91, 92 described herein provide an additional level of safety so that tissues are not unintentionally damaged from oversupply of electrical energy or unbalanced charges of energy. Or, they reduce or eliminate risk from use of non-approved stimulators, leads or electrodes (off-the-shelf components without the proper safety measures in place) to protect patients from an accidentally occurring overvoltage or over-current. The solution described herein is incorporated in the current path from the EPG to the injectable electrode and as such captures any electrical over-voltage or over-current to protect patients.

Over-voltage protection and over-current protection may be very specific for a patient, an anatomical location or a tissue target. For example, the skin of the human male lower back is relatively thick and provides a different electrical coupling than the comparably thinner skin of a human female arm. The electrical coupling between the EPG and the subcutaneously placed collector portion of the injectable electrode determines in part how much current may reach the deeper nerve 51 target within the body. By utilizing different levels of either over-voltage or over-current protection, the same EPG is used for different patients by (1) optionally limiting the maximum voltage and/or current supplied by the hardware by software setting inside the EPG and (2) furthermore utilizing the external hardware described herein to ensure that patients don't accidentally overstimulate a specific nerve. Different levels of protection for voltage levels can range from (a1) zero to ten volts (0-10V), (b1) 0 to 20V, (c1) 0 to 30 V, (d1) 0 to 40 V, (e1) 0 to 50 V, and so on in a linear fashion to a final range of 0 to 100 V. Likewise, the ranges may be in a geometric line for (a2) 0 to 10 V, (b2) 0 to 20 V, (c2) 0 to 50 V, (d2) 0 to 100V. These voltage limitations are embodied in different patch electrodes with particular colors, markings or shapes corresponding to a specific maximum voltage (e.g. 10V limited electrode patches as blue, 20V limited electrodes patches as green, and 50V limited electrode patches as purple).

Over-voltage protection at the extracorporeal stimulator level prevents overvoltage and over-current at the stimulator level, specifically on the inside of the stimulator. Circuitry shunts over-voltage to ground or shunts over-voltage between two or more active lines. In various embodiments, this is achieved with two antiparallel diodes as shown in the schematics in FIGS. 13A, 13B and 13C. FIG. 13A shows a basic over-voltage protection designed into the EPG itself. FIG. 13B is similar to 13A, but with a capacitor added in line with the two diodes. FIG. 13C is similar to 13A, but with diodes facing each other in 13A and away from each other in 13C which electrically results in the same sum of breakthrough voltages.

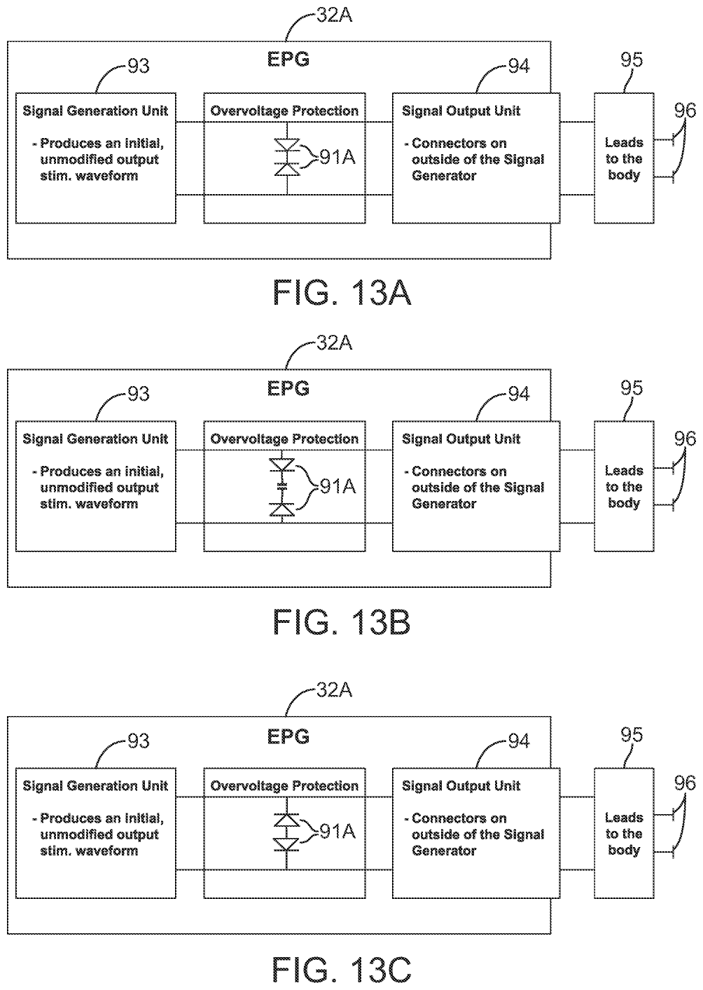

The second location to implement a voltage overprotection is at the interface location between the leads and the EPG. FIG. 14 is a schematic which illustrates this approach.

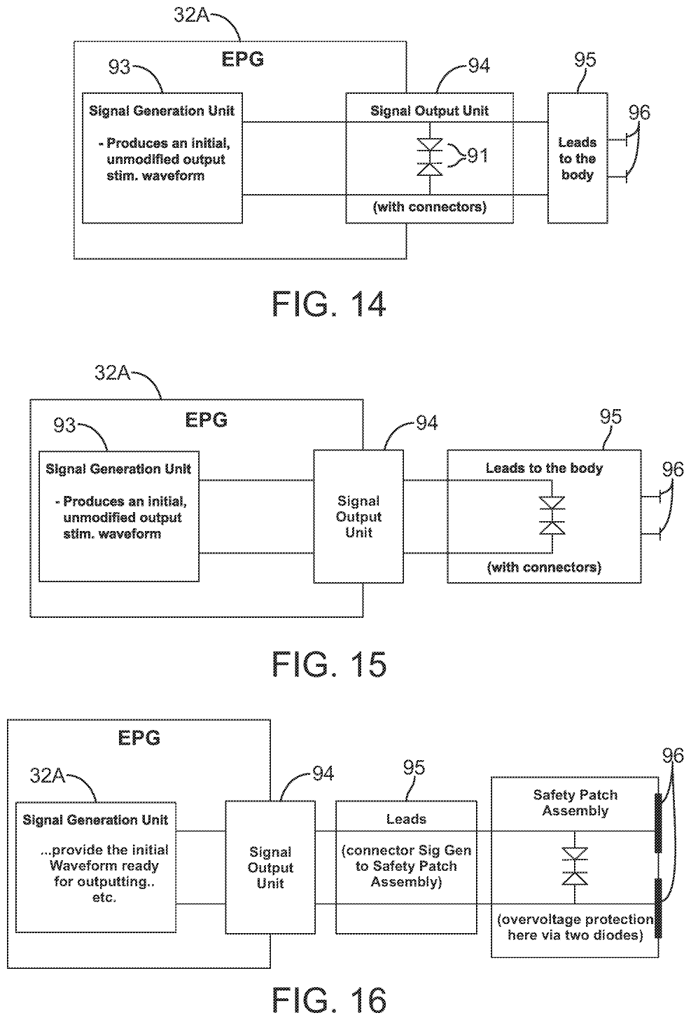

The third location to implement a voltage overprotection is at the interface location between the leads and the EPG, that is, at the extracorporeal lead level. FIG. 15 illustrates this approach.

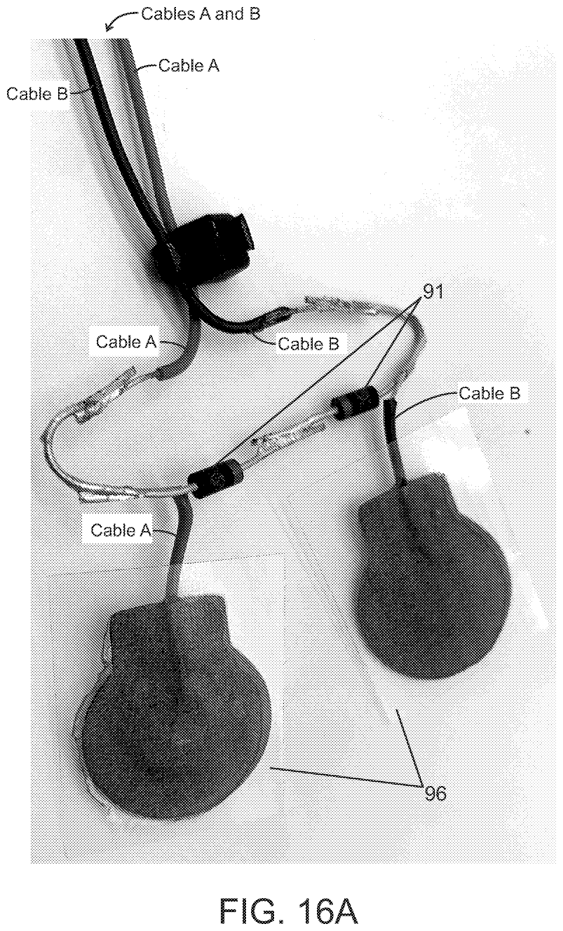

The fourth location for over-voltage protection is at the extracorporeal electrode level. This is analogous to the one before, that is, transferring the over-voltage protector from the lead to the electrode versus a counter electrode. FIG. 16 is a schematic showing basic over-voltage protection as part of the safety patch assembly comprising two diodes which are anti-parallel to each other and, as a group, are in parallel to the patient, thus allowing over-voltages to shunt away from the patient by short-circuiting through the two diodes. FIG. 16A is an image of a prototype system similar to the schematic system shown in FIG. 16. This is one embodiment of a prototype of an over-voltage protection patch assembly. It has two cables (A, B) to the EPG and two corresponding patch electrodes 96. It features two anti-parallel diodes 91 limiting the maximum voltage that can reach the patient to 10 V. Any voltage higher than 10 V will shunt through the diodes that are part of the patch assembly.

Similar to over-voltage protection, over-current protection may also be part of the present system. One simple over-current protective device is a fuse, in line with the primary current path from the output of the signal generator through the connecting wires, cable, connectors, electrodes to the (fully or partially) implanted electrode-lead combination (such as an injectable electrode) to the target nerve and back. However, this approach is limited by its single-use feature. Additionally, conventional fuses that operate about the maximal allowable currents for nerve stimulation are not often available. The system may employ a passive, fixed over-current protection scheme. Another approach is by way of incorporating a current-limiting diode (CLD), often also referred to as a constant current diode or current-regulating diode, as a series component. This can be easily placed anywhere within the signal generator, at one of the output terminals, or into the injectable electrode itself. CLDs have predetermined maximal thresholds. This means that over-current thresholds cannot be dynamically set after the CLD-based system is installed, preventing “hot-swapping” the same stimulator between applications with different current requirements.

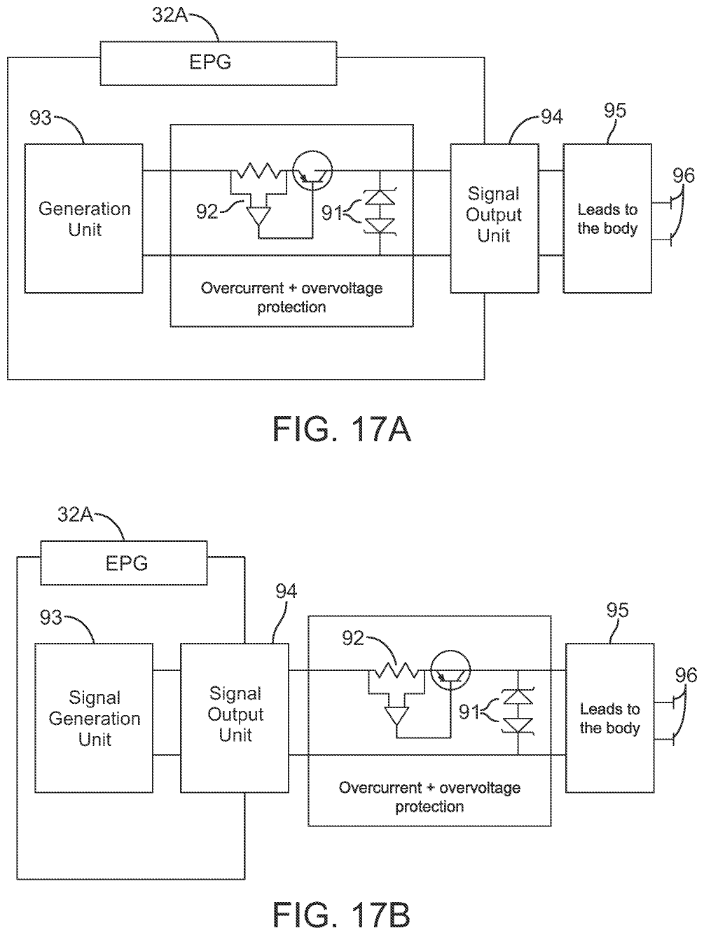

FIGS. 17A and 17B depict an EPG with an over-voltage and over-current protection unit (91 & 92) within the EPG (17A) and outside the EPG (17B).

Another embodiment employs the use of an active feedback monitoring system to regulate and modulate the current output as a means of over-current protection. If a glitch in the signal generation unit produces a higher than nominal voltage that exceeds current compliance, the active feedback system immediately decreases the conductivity of the transistor Q2 as in FIG. 12. The strength of this approach is that the current can be monitored as well as kept at nominal values. The active feedback circuit is also dynamically tuned to achieve different levels of current compliance using the same device as in FIGS. 17A and 17B.

Parts of the over-current protection include: (a) a signal generator 93, (b) leads 95, connectors, adapters between the signal generator and the patch electrodes 96, (c) a fuse or other over-current protector 92 (e.g. current shunt, integrated circuitry, etc.) and (d) an implanted injectable electrode 1 near the target nerve 51 (as shown in FIGS. 18 & 19. Fuses can include, without limitation, thin wires, wires that disconnect temporarily due to bending under heat, integrated circuits, and/or current shunts. An alternative path to ensure over-current is to provide a secondary shortening path that is at a much lower impedance, thereby being both, a over-voltage protection as well as an over-current protection. Overcurrent protection at the extracorporeal stimulator level is similar to the over-voltage approach described above, but in this case utilizing the fuse or equivalents at the stimulator level. Overcurrent protection at the extracorporeal (outside-the-body) lead level is similar to the over-voltage approach described above, but using the fuse or equivalents at the outside-the-body lead level. Overcurrent protection at the extracorporeal electrode level is similar to the over-voltage approach described above, but utilizing the fuse or equivalents at the outside-the-body-electrode level.

### Percutaneous Embodiments

In certain embodiments, an injectable electrode is injected near a tissue target but has an external tail 99 (shown in FIGS. 18 & 19) remaining outside the skin (“percutaneous injectable electrode”) with a bodily structure inside the body. This has advantages by being removable more easily than a completely injected injectable electrode which must be found (e.g. via ultrasound or fluoroscopy guidance) and then grabbed with or hooked with a removal device or with an open cut down and forceps). The percutaneous injectable electrode further has the advantage of conducting higher levels of current from the outside of the body to a deeper (e.g. neural) target structure. It also can be used as a sensing or listening device, allowing for the recording of neural signals such as electrically evoked compound action potentials (eCAP) or electronystagmographic (ENG) from the body or electromyographic (EMG) data to facilitate training or running closed-loop neuromodulation circuitry. The large surface area of the injectable electrode interfacing with the neural or muscular tissue of interest allows for a low impedance from the biological tissue into the metal wiring which in turn, when connected to an input amplifier on the inside or outside of the body allows for a low noise sampling and recording of biological signals from the live body. Providing this large surface area interface near a nerve or muscle target and thereby a low input impedance on that distal end, all while providing essentially a zero output impedance on the proximal (outside the body) connecting end permits short term (<30 day or <60 day or more) recording of ENG, EMG or eCAP data from a patient to train a neuromodulation algorithm with artificial intelligence and large data models to provide better stimulation algorithms for neuromodulation applications. The injectable electrode can be removed by grabbing and unzipping it with either a device or just with gloved hands to carefully pull and thereby remove the device using the unzipping techniques described in the '007 application. It differs from other commercially available percutaneous systems in several ways including, without limitation, that the coated or uncoated portions can self anchor without tines, hooks or sutures inside the body near a tissue target. Likewise additional anchors may be placed as anchors and/or to provide additional strain relief via unzipping at locations between the target location and the skin as well as right underneath the skin or in close proximity to the subcutaneous tissue or additional passage points in fascia tissue, for example.

The percutaneous injectable electrode has additional slack when compared to current temporarily placed wired systems that interface with neural structures inside the body and thereby offers an additional safety factor against unintentional removal by pulling of the externalized portion of the percutaneous injectable electrode. Furthermore, by leaving only a much shorter portion of the overall percutaneous injectable electrode on the outside of the body, the risk of such a device being pulled out is greatly reduced when compared to the peripheral nerve evaluation leads. The latter for sacral interfacing are neither self anchoring nor unzipping, nor can they form anchors during the injection process at locations that the physician decides to have (such as e.g. ½ the distance from the sacral foramen to the skin and then again in the subcutaneous tissue) before exiting the skin. There is great benefit to the patient of having at least 70% of the total length of the percutaneous injectable electrode be located below the skin and less than 30% of the total length be located outside the skin. In various implementations, it may be even more favorable to have only approximately 1 to 29% of the total length of the device be located outside the body, or even less for an average outside the body length of 1 to 10% or even only 1 to 5%. In order for the system to provide a reliable connection to the EPG while minimizing risk of the EPG pulling on the injected portion, the electrodes on the outside of the body has additional safety options and/or features.

### Additional Aspects of the Percutaneous Injectable Electrode

The percutaneous injectable electrode connects to the EPG by one or more of various mechanical connections.

One embodiment of such a connection comprises a non-stick portion and a sticky portion. The nonstick portion is overlaid with the externalized portion of the percutaneous injectable electrode, while the sticky portion (which may not conduct any or may conduct less current) is placed on the skin for mechanical protection and adhesion of the outside electrode to the body. The sticky portion surrounds the non-sticky portion akin to a ring with a non-sticky center portion to overlay the external tail of the percutaneous injectable electrode while providing protection against the elements to the externalization point. The conductive non-stick portion may be connected to a wire or pin to connect the external power source. In the embodiment shown in a schematic section view in FIG. 18, an electrically conductive bridge is formed by a conductive metal band 98, here a BAND-AID®-like device 97 which on its body-facing side has an outside-the-body electrode which then connects to a from-the-body-away-facing side, to offer a transfer of electrical current from below the device to above the device. Such a device shields the percutaneous injectable electrode at the exit point from the elements and allows the reversible placement of a secondary electrode or electrically conductive device with a stimulator on-top of the primary device 97 as described. A schematic view of a device with pass-through conductive metal band 98 is shown in FIGS. 18A and 18B. FIG. 18A shows the bottom of the device and 18B shows the outside facing side of the device. The band on the outside connects to an electrode on the EPG which is placed over it.

In other embodiments an insulating coating is placed on the skin 37 before placing the conductive device shown in FIGS. 18 and 19, meaning there is an insulating layer between the skin and the injectable electrode. The insulating layer is optional because the voltages needed to stimulate a cutaneous sensation are much higher than the voltages required to stimulate a nerve with the injectable electrode. That is, additional embodiments similar to FIGS. 18 and 19 can also have an additional insulating element placed on the skin (e.g., tape) which prevents current intended for the injectable electrode from stimulating the skin too.

Instead of the conductive metal band passing from below the device to the top, in one embodiment there is metal interwoven, electrically conductive tape or a conductive hook and loop device (such as Velcro®) such as one available from VETCO, Bellevue WA. This can be achieved with either (A) the typical combination of the loop side and the hook side, or (B) only the loop side being made with metal wires interwoven into the conductive loops. This is a mechanically flexible interface that electrically presses into and around the injectable electrode, thereby being shock and acceleration resistant. In other embodiments, the injectable electrode is sandwiched between the loops and the hooks or the loop side is placed on top of or below the injectable electrode and then secured with the rest of the device backing. Either configuration produces a mechanically reliable interface to the injectable electrode to allow a small electrical impedance between the electrically conductive device and the injectable electrode.

In many different kinds of embodiments, then, the electrical connectors are selected from the group consisting of metal bands secured in an adhesive, complementary conductive hook and loop strips, of several clips may be used such as snap on/in buttons, magnetic clips, spring loaded push pin buttons, spring loaded connectors, snaps to pins, and electrically conductive adhesive tape. In the latter connector, the back of the patch electrode assembly has a sticky portion that the EPG adheres to without a button which joins exposed metal on both the EPG and the patch electrode assembly).

In other embodiments, the conductor band holds voltage limiting or current limiting components as described herein.

In one embodiment of the device herein, a loop is formed during injection, that is, the end of the percutaneous portion is implanted in the skin to create an additional anchor when the subcutaneous tissue attaches to the electrode. This loop creates a better anchor of the percutaneous injectable electrode at the exit point, the device's tail may be reinserted right next to the exit point and thereby cause a secondary anchor point below the skin. Coated and uncoated regions of the percutaneous injectable electrode facilitates this double “exit and re-entry” in such ways that only the uncoated region is externalized to the body and the exit points only see coated regions, or variations with one of the exit points having coated and the other one an uncoated portion crossing the skin. A special re-introducer tool also may be a needle with a half-moon shaped, blunt cut-out to enable the re-injection of the external tail end into a nearby location with minimal risk of cutting the percutaneous injectable electrode during the reinsertion step. The injectable electrode can be reintroduced using the same needle used for placement by re-feeding the end of the device into the needle, or using a modified needle with a beveled edge and cutout, such as a Coude needle. (Epimed, Johnstown NY). This placement is shown in FIG. 19.

In cases where the original placement needle is used, the process of re-feeding and subsequent introduction into the body may be facilitated by the placement needle's blunt tip with a greatly reduced risk of unintentional bending, compressing or cutting of the injectable electrode or components of the injectable electrode. The steps for utilizing this approach are (1) using a sharp trocar (with or without presence inside the placement needle) to create a puncture hole in the skin, then (2) re-feeding the external tail 99 of the injectable electrode into the placement needle and (3) injecting the placement needle with injectable electrode present into the new puncture hole, (4) utilizing the deployment pin to fully inject the remainder of the injectable electrode into the body of the patient and form, when applicable, a new bunched anchor/irregular shape 8 in the second, new puncture hole prior to withdrawing the placement needle from the patient and covering the two puncture needle holes with wound dressing.

### Magnetic Resonance Imaging Compatibility

Helical wire rope structures for implantation have many advantages over previous devices and are known to exhibit lower magnet resonance imaging (MRI) artifact and heating than their uncoiled/non-helical counterparts. The presence of gas filled pockets within devices introduces local imaging artifacts. Described here are methods to modify injectable electrodes to reduce MRI artifacts. Intermittent and porous coating enables this. Coating is applied to mechanically hold the bulk of the injectable electrode from fine wire strands together and limit the amount of strand movement as well as partial separation of small loops of strand from the bulk. The coating, however, prevents an immediate and easy filling of the structure with interstitial fluid once injected into a body, or during a pre-filling with fluid inside the delivery cannula and prior to the actual deployment. While the inner core is hollow to allow collapsing of the injectable electrode during a subsequent optional removal, the hollow core likewise functions as a fluid path to optionally fill the injectable electrode with saline or sterile water or other liquids prior to deployment. A dry injectable electrode has been demonstrated to induce more MRI artifacts than a wetted injectable electrode. While the hollow core allows the removal of air from the overall injectable electrode inside the needle or once placed into a body, any applied coating on the outside of the rope of strands hinders the intrusion of any liquids for an extended period of time.

If there is a need for a patient (who earlier has received an injectable electrode placed into their body) to undergo an MRI procedure then it is advantageous for the entire injectable electrode to be filled with interstitial fluid or at least sterile saline or water from the deployment procedure earlier, or a mix of the pre-loaded liquid and interstitial fluid.

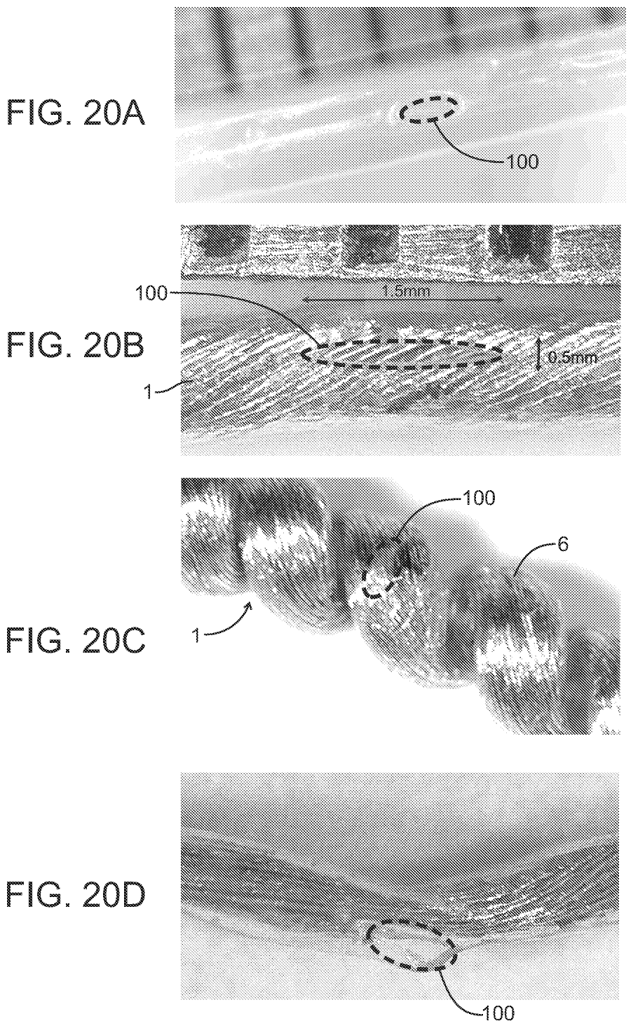

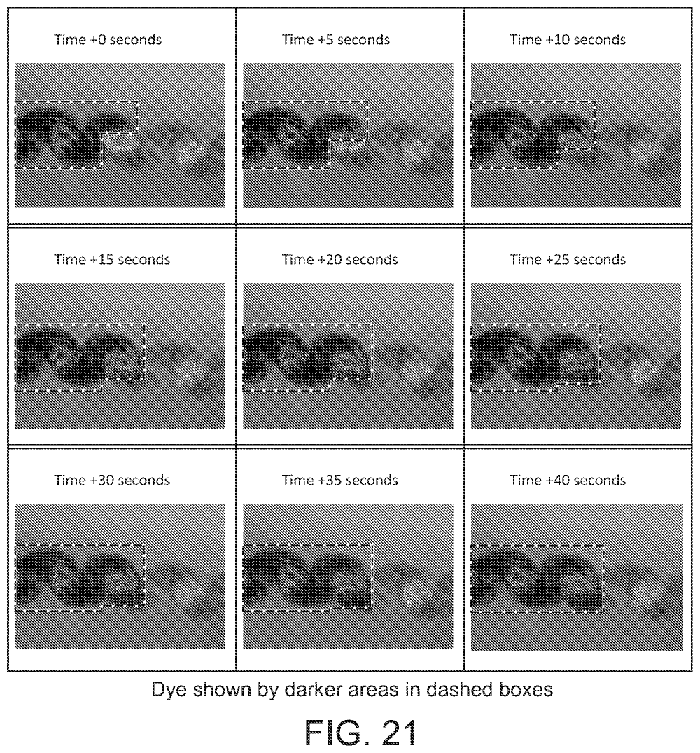

To further aid with the removal of air from the injectable electrode, or speed up the fluid ingression, gaps or cuts or holes in the coating may be added intentionally during the manufacturing process of the injectable electrode (FIGS. 20A-D). In FIG. 20, an injectable electrode is shown with intermittent coating approximately at the middle of the device. Fluid ingression into the coated regions is permitted not only from both ends, but also from the middle. More locations for fluid to ingress into the rope structure formed from strands allows for the entire injectable electrode to quickly and efficiently wick with either fluids prior to placement or interstitial fluids thereafter. In another embodiment, the coating may be continuous or intermittent from end to end, but is perforated along its length in one or more locations. Perforating the coating enables the coating to retain the wire strands in place but limit their ability to move relative to another outside of the designated electrode or designated cell-ingrowth areas, while permitting the inflow of interstitial fluid or other liquids such as before, during or after injection into the body. Perforation of the coating may be done with the same, or with different sizes of holes. Holes 100 (or gaps, perforations or stabs) may be added during the manufacturing process or may be added just prior to deployment. Generally, holes, gaps, perforations, or stabs are not to be placed at locations where the device would likely be near a nerve structure not intended to be electrically interfaced with (stimulated or blocked). In other words, holes and other forms of opening in the coating enabling a faster liquid inflow have the potential to function as a current bridge to nearby structures. As a result, holes would be at different locations for different target-optimized injectable electrodes. For example, an injectable electrode intended for a use in the neck may have fewer or no additional holes nearby the nerve-interfacing electrode (on the side more distal to the skin) with the understanding that there would be additional time needed for the wicking of said wire structure to be accomplished inside a body, but with the benefit that other nerve structures nearby the target structure in deep tissue of the neck would not co-activate or co-effect said nearby structures. Another embodiment of the perforated injectable electrode has the holes run in a spiral around the coating of the rope in such a way that, once wound around the mandrel, the holes are all facing inward towards the mandrel and thereby away from off-target structures that are not meant to be electrically interfaced with due to small current bridges that said holes are causing. This approach utilizes the effect that the interfacing electrode end is sitting right on (or very close to) the target structure, bunched up against the target structure and thus at a very small distance to said target structure, all while the holes permitting the influx of ionic liquids such as interstitial fluid are at least the distance of the coated rope away from any off-target nerve structure the injectable electrode may be passing by once placed inside a body. The second order spatial differential describing the activation probability dictates that distance effects the probability of electrical interfacing in the second or third order. A point source, such as a small hole in the coating, is deploying current densities that will drop off to the power of 3 with increasing distance, thus small holes (e.g. between 10 um and 100 um, or between 101 um and 200 um or between 201 and 500 um), especially when facing inward towards the center line of the injectable electrode, are much less likely to activate nearby neural structures when the injectable electrode is utilized to stimulate (and activate) a target structure nearby the distal interfacing end. FIG. 21 shows images showing fluid ingress in five second increments over 40 seconds. The darker portions in the boxes with dotted lines show the ingress of fluid inside the coating of the injectable electrode.

Other modifications to device/coating to improve wetting include (1) wicking agents (like PEG) or the addition of hydrophilic materials or coatings within the structure (such as polyvinypyrrolidone) to hasten and make wetting more complete; (2) during the manufacturing process, wetting agents (such as PEG) or agents increasing the hydrophilic behavior and lowering the surface tension are applied prior to skeining the strands into the wire rope as well as prior to winding the rope from the skeined wire rope. Likewise, wetting agents and similar may be applied to the finished rope prior to coating by e.g. heat-shrink tubing or may be applied prior or during the coating with a dip-coating or spray coating agent. Dry wicking structures (such as wickable strands in addition to the electrically conducting strands) may be woven into the skeined strands prior to or in parallel to the twisting of the strands to form the rope. (3) Lastly, small ropes of parts of the entire set of strands (e.g. 25 out of a total of 100) may be woven or twisted into one rope, the process repeated four times, to allow for smaller wicking channels inside each rope and the four ropes of 25 strands may then be twisted together to form a larger rope prior to coating the structure of 4 ropes of 25 strands each, thereby creating a large 4-strand rope of smaller ropes. This structure has smaller and larger wicking channels of different diameters that allow for an aided fluid ingression, the larger diameter channels between the 4 sub-ropes allowing fluids to penetrate faster and then the smaller channels allowing for a slower but complete fluid penetration. Likewise, 3 ropes of e.g. 30-34 strands can be wound, thereby allowing three sub-ropes to be braided into a larger rope structure of again 100 strands in total prior to coating the braided structure with e.g. heat-shrink tubing. The braided structure again has larger holes and channels inside the coating, permitting a faster and more efficient wicking of the structure.

In one embodiment, the injectable device (e.g. injectable electrode) is delivered in a transfer cannula or a placement cannula. Prior to injecting the device into a body, it is primed with a liquid either during the placement or inside a transfer cannula to provide abundant liquid in close proximity to the device and substantially more than may be present if a dry device were to be injected into a body, thereby permitting a faster wicking of the entire device than without the priming.

Further modifications to reduce MRI artifact may include: high inductance at high frequencies which increases effective impedance, and high impedance reduces energy flow; increase of the pitch of the windings; thicker insulation to decrease energy pickup during MRI; antenna (lead conductor) length can increase or reduce energy pickup and transfer to the body's tissue (examining half (fractional) wavelength of expected machine use (1.5T, 3T, etc.) can help with choosing optimum length to reduce energy pick up/transfer; reduced energy can reduce heating, and induced stimulation; while some embodiments of an injectable electrode may be formed from one continuous strand for the entire length, for MRI de-tuning shorter strand portions may be interwoven or skeined together and electrically and electromagnetically forming a different antenna function); welding, soldering or crimping together two (or more) ends or along different locations of the strands changes the antenna characteristics of the rope of the injectable electrode including breaking standing waves; increasing non-coated electrode area spread the picked up energy over a larger surface area to reduce tissue heating; breaks in average pitch (sudden increases and decreases in conductor pitch and diameter) to break up standing waves in the conductor, reducing or eliminating energy pickup and transfer; weaving one larger diameter strand (e.g. 50 um, 75 um or 100 um) at one or more intervals into a rope of small diameter strands (e.g. 25 um) to change impedance characteristics to disrupt the formation of standing wave functions inside the rope and injectable electrode, especially when coating is applied.

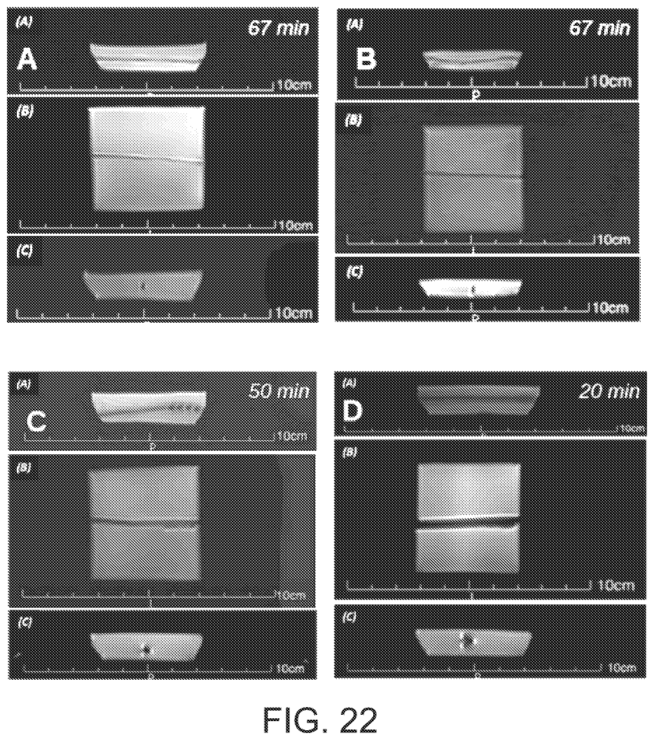

MRI 3T testing was performed in agar gel phantoms as shown in FIG. 22. An uncoated injectable electrode or a portion of uncoated injectable electrode or a thoroughly wetted injectable electrode shows the smallest artifact in an agar gel. A dry coated injectable electrode still only shows a very small MRI artifact, slightly more visible than the uncoated injectable electrode, but significantly less artifact than traditional leads used in neuromodulator today. Furthermore, the electrode portion, formed from the uncoated injectable electrode part, has a much smaller tendency to cause MRI artifacts (see FIG. 22C below). The injectable electrode (A) uncoated and (B) coated regions displayed little to no artifact, in comparison to standard clinical electrodes with either (C) four or (D) eight contacts, which exhibit mild (C) to significant (D) artifacts at shorter imaging intervals compared to the injectable electrode. Note the four electrode contacts in (C) showing up as four separate artifact inducing sections and how the eight electrode contacts cause a continuous large artifact visual. Negligible temperature increases were seen in all samples (28° C. threshold). Three views of each gel are provided (axial, coronal, sagittal) with a zoomed in coronal view of each shown on the left to highlight artifact causing distortions around MRI images of each of the devices.

### External Pulse Generator EPG

In various embodiments, the EPG is a voltage or current controlled analogue or digital pulse generator comprising two or more electrode contacts to interface with one, two or more partially or fully implanted injectable electrodes.

In one embodiment, an EPG provides a voltage controlled, symmetrical charge balanced waveform between two electrodes that are on the outside of the body.

For fully implanted injectable electrodes, devices that are below the skin end to end, the EPG may interface with the body through two or more hydrogel electrode patches. These hydrogel electrode patches receive their voltage signals from the EPG and transfer them to the skin in close vicinity to the tail end of at least one injectable electrode, e.g., located in the subcutaneous tissue.

Such a two-hydrogel patch EPG may utilize one patch electrode above one injectable electrode tail and the second patch electrode touching the skin of the patient distant to the injectable electrode, thereby completing the electronic circuit. Alternatively, the EPG may interface with two injectable electrodes placed into the same patient, by placing one patch electrode over the (subcutaneous) tail end of one injectable electrode and the second patch over the (subcutaneous) tail end of a second injectable electrode. In another embodiment, a three-hydrogel patch EPG interfaces with two injectable electrodes placed into the same patient, by placing one patch electrode over the (subcutaneous) tail end of one injectable electrode, a second patch over the (subcutaneous) tail end of a second injectable electrode and the third patch electrode touching the skin of the patient distant to either of the two injectable electrodes, thereby completing the electronic circuit with three potential current paths, one between the two injectable electrodes directly, the second one between injectable electrode 1 and the distant return and the third one between injectable electrode 2 and the distant return, allowing for current steering to reduce, limit and mitigate otherwise potentially present stimulation of nearby neural tissues such as nerves running in parallel to one of the injectable electrodes in cases where the actual target nerve has e.g. at least one other nerve running nearby. Current steering by field shaping the activation energy away from an unwanted location and concentration towards the target location become possible with such a 3 pole- or three patch electrode-setup.

Other elements incorporate over-voltage and/or over-current protection be integrated in the EPG via software and/or hardware.

In other embodiments, the EPG is programmable and controllable via APP on an outside programmer or control unit. In additional embodiments, the EPG is configured to communicate via near-field-communication (NFC) or Bluetooth or other internet protocols and radiofrequency or optical signals with an additional controller or programmer.

The EPG can be configured to record bodily and bio signals such as voltage indicating heart rate, breathing and other muscular activities via EMG or other electrical measurements. The EPG may have embodiments which record metabolic or patient activity levels by measuring acceleration and orientation to assess sleep/wake cycles and activity levels throughout the day. In embodiments which record signal, the EPG may be configured to use these signals to trigger different waveforms at different times to provide different therapy effects, including those based on inferred patient activity or time of day or schedule during the day, week, or month. In these embodiments the EPG can be configured to communicate the recorded bodily and/or bio signals to an outside interface device (via NFC, Bluetooth or other), to allow external processing and optimizations in said outside interfacing device or in a secondary connected computer such as artificial intelligence in the cloud, to select optimum waveforms to be generated by the EPG and which can be triggered at different times throughout the day, week, month and be programmed at the same or a different point in time with the algorithm which bio signals or bodily signals or environmental signals (temperature, positioning, orientation, barometric pressure, accelerations the EPG is subjected to etc.) may require a different waveform. An EPG interfacing via metal to metal connection to one or more percutaneous injectable electrodes can be configured to use voltage recordings from deep inside the body as part of a sensory data set to drive decision making algorithms.

The EPG can be configured to use temperature, humidity or barometric pressure in the vicinity of the EPG to assess if a stimulation event is needed and initiate electrical stimulation based on changes in these environmental signals with or without incorporating activity levels based on, e.g., a step count of a patient. Many chronic conditions have symptoms that are triggered by environmental factors such as changes in temperature, humidity or barometric pressure that can be captured by various embodiments of the EPG for use as either a single trigger or in combination with other factors to assess the need for the initiation of electrical stimulation. An application of such changes in temperature, humidity or barometric pressure are for patients whose perception of pain changes with altitude, short term changes in weather (cold front/warm front) or seasonality of weather events. Environmental and bio data collected by some embodiments of the EPG is transferred to a smart mobile device allowing further data capture, alignment, processing, upload to and download from the cloud and interfacing with AI processing of the data as well as patient specific feedback about increases and decreases in patient reported symptoms or therapy outcomes. The clinician and/or patient may utilize these data and optionally AI assisted analysis and decision making matrices for optimized choices of waveform parameters, waveform application and cessation of waveform application.

Over-voltage and/or over-current protection may be part of the EPG, or it may be part of the accessories supplied with the EPG.

Similar to the EPG interfacing with a single injectable electrode, for percutaneous injectable electrodes the EPG can interface with the skin through one or more hydrogel electrode patches as well as one or more metal to metal connections to the injectable electrodes protruding from the inside of the body out through the skin. The hydrogel electrode patches receive their voltage signals from the EPG and transfer them to the skin as an optional counter electrode when placed directly onto the skin. To interface with the percutaneous injectable electrodes, metal to metal, carbon to metal or different means can also be used besides hydrogel patches to avoid voltage loss from a hydrogel to the percutaneous tail end of the injectable electrode on the outside of the body.

Similar to the EPG interfacing with two injectable electrodes, other embodiments of the EPG can be configured to comprise connection points to either only the two injectable electrodes, or also to a third counter electrode provided as a hydrogel patch to connect to the skin of the patient to allow the feature of current steering by enabling three potential current paths.

### Further Aspects of Configuration of Wire Rope and Injectable Electrode

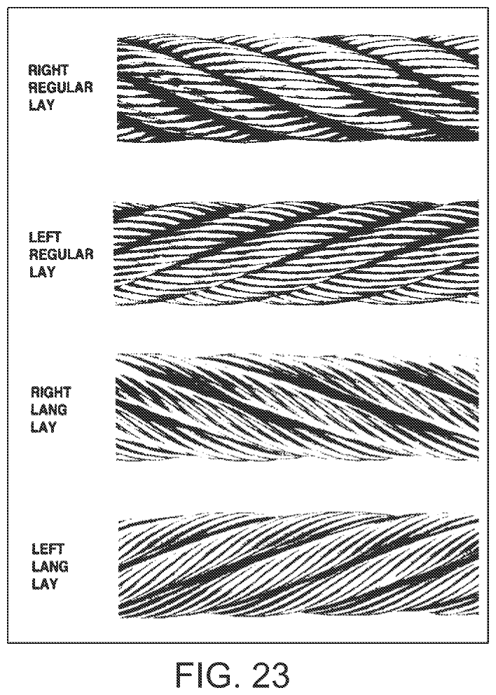

In rope making, the individual fibers of the rope can be twisted together and wound in one of four ways. The wires in each strand are twisted in the opposite direction as the strands in the rope for a regular lay, and in the same direction in a lang lay, as shown in FIG. 23.

In one embodiment the injectable electrode is in a Lang Lay configuration with the primary benefit that it is better for push-pull applications, is more flexible, and has greater fatigue resistance, but should be wound tightly and fixed at each end or it can come unwound. Lang lay means that the secondary helical winding is in a direction the same as the primary direction of twist of the parallel strands. In regular lay, the direction of winding and twisting are opposite. A regular lay helical wire structure electrode will be elongated per coil and therefore have a longer overall length due to a reduced overall diameter when wound around the same diameter winding core. Regular and lang lay injectable electrodes have different injection kinetics. The lang lay will be more pushable (i.e., longer overall device can be pushed through a cannula without physically jamming within the cannula. The deflection force required for formation of a bundle within tissue is lower for the lang lay, so that lang lay will have a greater angle of deflection than that of regular lay when injected into tissue with equivalent durometer/stiffness providing the same deflection force.

FIG. 23 shows the individual parallel strands in a lang lay and a regular lay configuration for both right-hand and left-hand twists. The lang lay configuration requires the secondary helical winding to be in the same direction (right-hand or left-hand) as the primary direction of twist of the parallel strands. In the regular lay configuration, the direction of winding and twisting are opposite. For the same number of parallel wires, diameter, and tight coil stacking approach, a regular lay helical wire structure electrode will be elongated per coil (3) and therefore have a longer overall length (4) due to a reduced overall diameter when wound around the same diameter winding core.

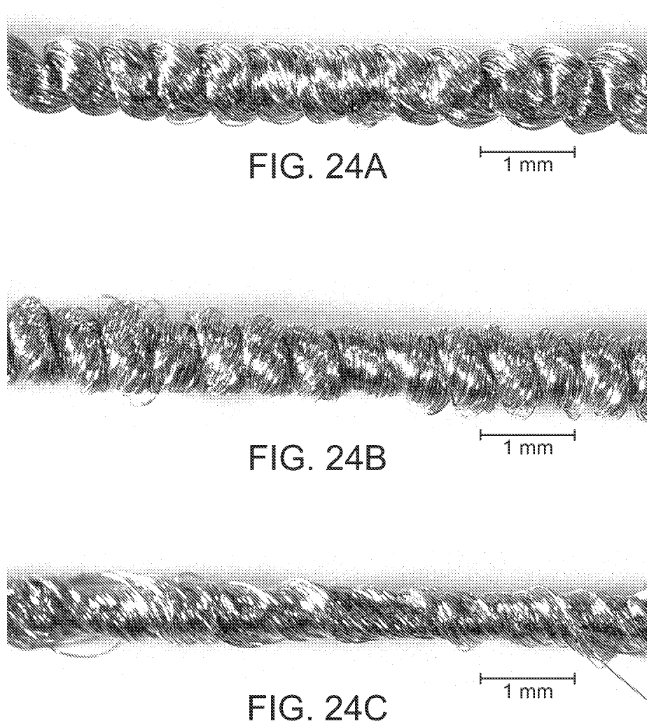

FIGS. 24A-24C are images of injectable electrodes with different configurations for twisting and coiling wire rope with a 1 mm scale bar. FIG. 24A is regular lay, 24B is a lang lay with twisting, and 24C is regular lay without twisting.

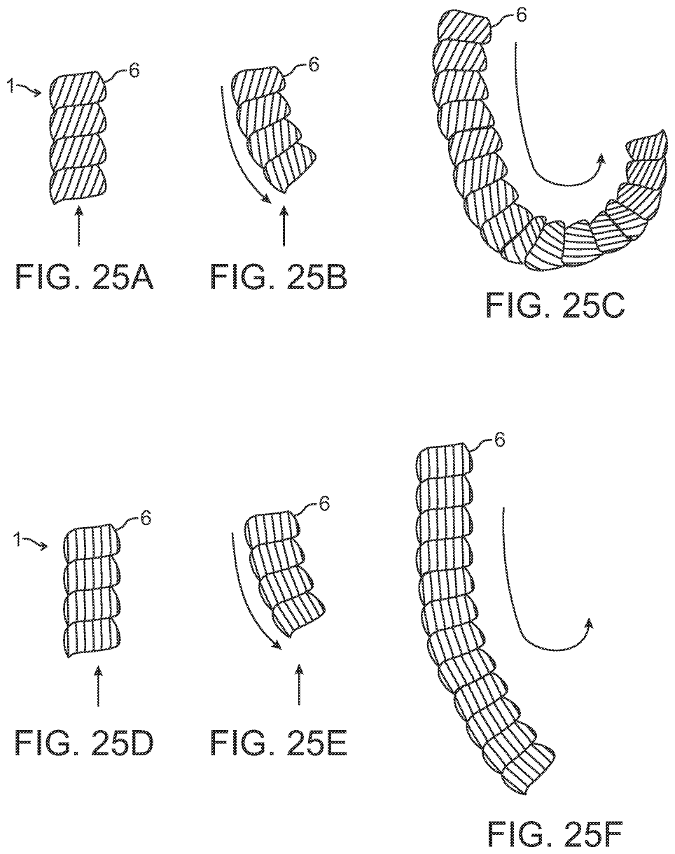

FIGS. 25A-25F show that lang lay (FIGS. 25A-25C) and regular lay (FIGS. 25D-25F) injectable electrodes have different injection kinetics. The lang lay structure is more pushable through a cannula without physically jamming within the cannula. The deflection force required for formation of a bundle within tissue is lower for the lang lay structure (25-C) compared to regular lay (25-F), such that a lang lay injectable electrode has a greater angle of deflection than that of a regular lay injectable electrode when injected into tissue with equivalent durometer/stiffness providing the same deflection force. These figures are not to scale.

1. A system for stimulating a tissue target in a body, the system comprising:
a helical wire rope structure electrode having a hollow core all along a length with first and second ends configured for loading into a dispenser in a substantially linear form all along the length for injection, and said helical wire rope structure electrode being further configured to deform from said substantially linear form into a plurality of irregular shapes in the body when injected, the plurality of irregular shapes including at least a first of the irregular shapes being located at the first end near a tissue target and a second of the irregular shapes being located in a subcutaneous area and the helical wire rope structure electrode configured to transmit electricity passively between said first irregular shape and said second irregular shape, and
an external pulse generator (EPG) for generating an electrical waveform, and configured to transmit electricity through an electrical path across the skin to the second irregular shape in the subcutaneous area.

2. The system of claim 1 wherein the EPG is configured to provide the waveform transcutaneously through at least two interfacing points to the helical wire rope structure.

3. The system of claim 1 further comprising at least one protective unit to prevent at least one of current or voltage above a safe level, the at least one protective unit including at least one protective unit located within one of the EPG and the electrical path from the EPG to the helical wire rope structure electrode.

4. The system of claim 1 further comprising a partial coating on the helical wire rope structure between the first and second ends, said partial coating comprising at least one discontinuity selected from the group of holes, gaps and perforations.

5. The system of claim 4 further comprising at least one wicking agent.

6. The system of claim 1 wherein the helical wire rope structure electrode comprises a wire rope comprising strands twisted in a first direction and then coiled in a directed selected from the first direction and a direction opposite the first direction.

7. The system of claim 3 wherein the at least one protective unit includes at least one protective unit having indicia indicating a particular limitation for at least one of the current and the voltage, the indicia including at least one indicia selected from the group of markings, colors, and specific shapes.

8. The system of claim 1 wherein the helical wire rope structure electrode, in its substantially linear form, has an outer diameter and an inner diameter, and a ratio of the inner diameter to the outer diameter is greater than 1:4.

9. (canceled)

10. A system for stimulating a tissue target, the system comprising:
a helical wire rope structure electrode having a hollow core all along a length with first and second ends configured for loading into a dispenser in a substantially linear form all along the length for injection of a portion of the length into a body, and said helical wire rope structure electrode being further configured to deform from said substantially linear form into at least one irregular shape at any location along the length inside the body for self-anchoring without tines, hooks or sutures when injected, the second end being configured to extend as an external tail beyond the skin, and the helical wire rope structure electrode configured to transmit electricity passively along all the length,
an external pulse generator (EPG) for generating an electrical waveform, and
a lead from the EPG and an electrical connection to the external tail.

11. The system of claim 10 wherein the EPG is configured to provide the waveform through at least two interfacing points to the helical wire rope structure.

12. The system of claim 10 further comprising at least one protective unit to prevent at least one of current or voltage above a safe level wherein the at least one protective unit includes at least one protective unit located within one of the EPG, at an interface between the EPG and the lead, within the lead, at a connection between the lead and the helical wire rope structure electrode, and within the helical wire rope structure electrode.

13. The system of claim 10 wherein the electrical connection comprises a conductive metal band.

14. The system of claim 13 wherein the conductive metal band is at least partially housed in an adhesive strip, and wherein the conductive metal band has one end that is configured for contact with the external tail, and another end that is configured for contact with the EPG.

15. The system of claim 10 wherein the electrical connection is a conductive clip connected to the EPG and is configured for attachment to the external tail.

16. The system of claim 10 wherein the electrical connection is a conductive hook and loop device.

17. The system of claim 10 further comprising a partial coating on the helical wire rope structure between the first and second ends, said partial coating comprising at least one discontinuity selected from the group of holes, gaps and perforations.

18. The system of claim 17 wherein the helical wire rope structure further comprises at least one wicking agent.

19. The system of claim 10 wherein the helical wire rope structure electrode comprises a wire rope comprising strands twisted in a first direction and then coiled in a directed selected from the first direction and a direction opposite the first direction.

20. The system of claim 12 wherein the at least one protective unit includes at least one protective unit having indicia indicating a particular limitation for at least one of the current and the voltage, the indicia including at least one indicia selected from the group of markings, colors, and specific shapes.

21. The system of claim 10 wherein the helical wire rope structure has an outer diameter and an inner diameter, and a ratio of the inner diameter to the outer diameter is greater than 1:4.

22. (canceled)
